SEQUENCE LISTING (1 sequence)

SEQ ID NO: 1 - RNA, 20 nt, synthetic construct
  source 1..20: mol_type = other RNA
ttgtgactgtaagtgttgga

(19) United States
(12) Patent Application Publication
(10) Pub. No.: US 2026/0295087 A1
(43) Pub. Date: Oct. 1, 2026

(54) METHODS FOR TREATING AN OCULAR DISEASE

(71) Applicant: The Johns Hopkins University, Baltimore, MD (US)

(72) Inventors: Allen O. Eghrari, Baltimore, MD (US); John D. Gottsch, Glencoe, MD (US); Jinchong Xu, Lutherville Timonium, MD (US)

(21) Appl. No.: 19/477,582

(22) PCT Filed: Apr. 25, 2024
(86) PCT No.: PCT/US2024/026291

§ 371 Date: Oct. 22, 2025

Related U.S. Application Data
(60) Provisional application No. 63/461,757, filed on Apr. 25, 2023.

(51) Int. Cl. A61K 48/00; A61K 35/30; A61K 38/39; A61P 27/02; C12N 5/079; C12N 9/22; C12N 15/11

(52) U.S. Cl. CPC A61K 48/005; A61K 35/30; A61K 38/39; A61P 27/02; C12N 5/0621; C12N 9/226; C12N 15/111; C12N 2310/20; C12N 2506/45; C12N 2510/00

(57) ABSTRACT

Provided herein are methods of treating an ocular disease in a subject in need thereof, the method comprising: (a) obtaining an induced pluripotent stem cell (iPSC) from the subject; (b) delivering a gene-editing agent into the iPSC, wherein the gene-editing agent alters a target gene of the iPSC; (c) differentiating the altered iPSC into a corneal endothelial cell; and (d) administering the corneal endothelial cell to the subject, thereby treating the ocular disease in the subject.

### CROSS-REFERENCE TO RELATED APPLICATIONS

This application claims priority to U.S. Provisional Patent Application No. 63/461,757, filed on Apr. 25, 2023, which is incorporated herein by reference in its entirety.

### SEQUENCE LISTING

This application contains a Sequence Listing that has been submitted electronically as an XML file named 44807-0454WO1_ST26_SL.XML.” The XML file, created on April 23,2024, is 110,595 bytes in size. The material in the XML file is hereby incorporated by reference in its entirety.

### TECHNICAL FIELD

The present disclosure relates to the area of generating an engineered corneal endothelial cell from induced pluripotent stem cells and uses thereof. In particular, it relates to engineering a corneal endothelial cell by using gene-editing agents to repair gene mutations in the cell.

### BACKGROUND

Fuchs endothelial corneal dystrophy (FECD), which affects 1 in 40 Americans, is characterized by progressive loss of corneal endothelial cells, thickening of Descement's membrane and deposition of extracellular matrix in the form of guttae. When the number of endothelial cells becomes critically low, the comea swells and causes loss of vision. The clinical course of FECD usually spans 10-20 years, often requiring transplantation of endothelial cells to restore vision and corneal clarity. Over the last several decades genetic studies have detected several genes, as well as areas of chromosomal loci associated with the disease. However, although keratoplasty has been successful at visual rehabilitation, graft rejection and lack of suitable donor tissue for transplantation continue to be impediments to reduce worldwide corneal blindness.

### SUMMARY

Provided herein are methods of treating an ocular disease in a subject in need thereof, the method comprising: (a) obtaining an induced pluripotent stem cell (iPSC) from the subject; (b) delivering a gene-editing agent into the iPSC, wherein the gene-editing agent alters a target gene of the iPSC; (c) differentiating the altered iPSC into a corneal endothelial cell; and (d) administering the corneal endothelial cell to the subject, thereby treating the ocular disease in the subject.

In some embodiments, the iPSC is obtained from blood of the subject. In some embodiments, the iPSC is a peripheral blood mononuclear cell (PBMC) originated iPSC.

In some embodiments, the gene-editing agent comprises CRISPR/Cas9 components. In some embodiments, the gene-editing agent comprises a guide RNA (gRNA), wherein the gRNA is targeted to the target gene of the iPSC. In some embodiments, the target gene comprises a COL8A2 gene. In some embodiments, the target gene comprises a L450W mutation in the COL8A2 gene. In some embodiments, the gene-editing agent alters the L450W mutation in the COL8A2 gene. In some embodiments, the gene-editing agent further comprises a PAM sequence. In some embodiments, the PAM sequence comprises a TGG sequence.

In some embodiments, the ocular disease comprises Fuchs endothelial corneal dystrophy (FECD), keratoconus, keratoconjunctivitis sicca (KCS). hepes virus infections, varicella-zoster virus infections. irido-corneal endothelial syndrome (ICE), pterygium, Stevens Johnson Syndrome (SJS), corneal ulcers, or bullous keratopathy. In some embodiments, the ocular disease is Fuchs dystrophy.

Also provided herein are methods of generating an engineered corneal endothelial cell, the method comprising: (a) obtaining an induced pluripotent stem cell (iPSC) from a subject; (b) delivering a gene-editing agent into the iPSC, wherein the gene-editing agent alters a target gene of the iPSC; and (c) differentiating the altered iPSC into an engineered corneal endothelial cell.

In some embodiments, the iPSC is obtained from blood of the subject. In some embodiments, the iPSC is a peripheral blood mononuclear cell (PBMC) originated iPSC.

In some embodiments, the gene-editing agent comprises CRISPR/Cas9 components. In some embodiments, the gene-editing agent comprises a guide RNA (gRNA), wherein the gRNA is targeted to the target gene of the iPSC. In some embodiments, the target gene comprises a COL8A2 gene. In some embodiments, the target gene comprises a L450W mutation in the COL8A2 gene. In some embodiments, the gene-editing agent alters the L450W mutation in the COL8A2 gene. In some embodiments, the gene-editing agent further comprises a PAM sequence. In some embodiments, the PAM sequence comprises a TGG sequence.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows images of corneal endothelial cells grown from induced pluripotent stem cells from blood cells from a subject.

FIG. 2A shows genes expressed in corneal endothelium but not in stem cell, confirming the cells have differentiated from stem cells into corneal endothelium.

FIG. 2B shows genes expressed in stem cells but not in corneal endothelial cells.

FIG. 3 shows images of iPSC-derived corneal endothelial cells showing the same characteristics as corneal endothelial cells from the eye.

FIG. 4 shows images of corneal endothelial cells grown from the blood of a patient with the L450W mutation in COL8A2 demonstrating decreased cell density and fewer interactions between cells.

FIG. 5 shows an exemplary schematic of the L450W mutation in COL8A2 gene and the correction of the mutation in induced pluripotent stem cells using CRISPR.

FIG. 6 shows images of corneal endothelial cells from gene-corrected iPSCs looking similar in number and appearance when compared to normal corneal endothelium.

FIG. 7 shows gene expression analysis results showing that the presence of L450W genetic mutation in COL8A2 affects gene expression, and CRISPR correction of the L450W mutation restores gene expression levels to a profile similar to normal cells.

FIGS. 8A-8B show image analysis showing that qualities of normal borders are missing when cells have the L450W mutation in COL8A2, and after gene correction, the cells create healthy borders with tight junctions.

### DETAILED DESCRIPTION

Provided herein are methods of treating an ocular disease in a subject in need thereof, the method including (a) obtaining an induced pluripotent stem cell (iPSC) from the subject; (b) delivering a gene-editing agent into the iPSC, wherein the gene-editing agent alters a target gene of the iPSC; (c) differentiating the altered iPSC into a corneal endothelial cell; and (d) administering the corneal endothelial cell to the subject, thereby treating the ocular disease in the subject. Also provided herein are methods of generating an engineered corneal endothelial cell, the method including (a) obtaining an induced pluripotent stem cell (iPSC) from a subject: (b) delivering a gene-editing agent into the iPSC, wherein the gene-editing agent alters a target gene of the iPSC; and (c) differentiating the altered iPSC into an engineered corneal endothelial cell.

Various non-limiting aspects of these methods are described herein, and can be used in any combination without limitation.

It must be noted that, as used in the specification and the appended claims, the singular forms “a,” “an” and “the” include plural referents unless the context clearly dictates otherwise.

As used herein, the term “administration” typically refers to the administration of a composition to a subject or system to achieve delivery of an agent that is. or is included in. the composition. Those of ordinary skill in the art will be aware of a variety of routes that may, in appropriate circumstances, be utilized for administration to a subject, for example a human.

For example, in some embodiments, administration may be ocular, oral, parenteral, topical, etc.

In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic). mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, a “cell” can refer to either a prokaryotic or eukaryotic cell, optionally obtained from a subject or a commercially available source.

As used herein, “delivering” or “gene delivery” can refer to the introduction of an exogenous polynucleotide into a host cell, irrespective of the method used for the introduction.

Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelles biocompatible polymers, including natural polymers and synthetic polymers; lipoproteins; polypeptides; polysaccharides; lipopolysaccharides; artificial viral envelopes: metal particles; and bacteria, or viruses, such as baculovirus. adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “engineered” refers to the aspect of having been manipulated by the hand of man. For example, a polypeptide is considered to be “engineered” when the polypeptide sequence is altered or manipulated. For example, in some embodiments, an engineered polypeptide comprises a sequence that includes one or more amino acid mutations, deletions and/or insertions that have been introduced by the hand of man into a reference polypeptide sequence. In some embodiments, an engineered polypeptide includes a polypeptide that has been fused (i.e., covalently linked) to one or more additional polypeptides by the hand of man, to form a fusion polypeptide that would not naturally occur in vivo.

Comparably. a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity.

As used herein, the term “subject” refers to an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

### Method of Treating an Ocular Disease

Provided herein are methods of treating an ocular disease in a subject in need thereof, the method including (a) obtaining an induced pluripotent stem cell (iPSC) from the subject; (b) delivering a gene-editing agent into the iPSC, wherein the gene-editing agent alters a target gene of the iPSC; (c) differentiating the altered iPSC into a corneal endothelial cell; and (d) administering the corneal endothelial cell to the subject, thereby treating the ocular disease in the subject.

As used herein, an “ocular disease” refers to a condition or disease that affects the human eye and visual system, wherein an ocular disease interferes with the ability of the eye to function properly and/or negatively affects the visual clarity of the eye. For example, an ocular disease can include, but is not limited to, conjunctivitis, dry eye, macular degeneration, diabetic retinopathy, glaucoma, cataracts, keratoconus, or Fuchs dystrophy. In some embodiments, the ocular disease comprises Fuchs endothelial corneal dystrophy (FECD). keratoconus, keratoconjunctivitis sicca (KCS), hepes virus infections, varicella-zoster virus infections, irido-corneal endothelial syndrome (ICE), pterygium, Stevens Johnson Syndrome (SJS), corneal ulcers, or bullous keratopathy. In some embodiments, the ocular disease is Fuchs dystrophy.

In Fuchs dystrophy, fluid builds up in the comea of the front of the eye, causing the cornea to swell and thicken. In some embodiments, Fuchs dystrophy can affect both eyes and cause vision to gradually worsen over years. Symptoms of Fuchs dystrophy can include eye pains, discomfort in bright lights, fluctuating eyesight throughout the day, halos and/or glares from bright lights, and blurry vision combined with poor contrast in colors. In some embodiments, treatments for Fuchs dystrophy can include eyedrops, eye ointment, corneal transplant surgery, and/or endothelial keratoplasty.

As used herein, an “induced pluripotent stem cell (iPSC)” is a type of pluripotent stem cell derived from adult somatic cells that has been genetically engineered to an embryonic stem (ES) cell-like state through the forced expression of genes and factors important for maintaining the defining properties of ES cells. Induced pluripotent stem cells can be derived from skin or blood cells that been reprogrammed back into an embryonic-like pluripotent state, wherein the iPSCs are enabled as an unlimited source of any type of human cell needed for therapeutic purposes. In some embodiments, an iPSC is obtained from blood of a subject. In some embodiments, the iPSC is a peripheral blood mononuclear cell (PBMC) originated iPSC.

As used herein, a “gene-editing agent” can refer to an agent that can target and bind to a specific sequence in DNA. In some embodiments, a gene-editing agent comprises CRISPR/Cas9 components. As used herein, the term “CRISPR” refers to a technique of sequence specific genetic manipulation relying on the clustered regularly interspaced short palindromic repeats pathway, which unlike RNA interference regulates gene expression at a transcriptional level. As used herein, a “Cas effector” or “CRISPR-associated protein” can refer to an enzyme or protein that uses CRISPR sequences as a guide to recognize and cleave specific nucleic acid strands that are complementary to the CRISPR sequence. A gene-editing Cas effector can associate with a CRISPR RNA sequence to bind to, and alter DNA or RNA target sequences. In some embodiments, the gene-editing agent comprises a gene-editing Cas effector.

In some embodiments, the gene-editing Cas effector comprises a Cas9 protein, a Cas13b protein, or a Cast3d protein. In some embodiments, a gene-editing Cas effector can be a Cas9 endonuclease that makes a double-stranded break in a target DNA sequence. In some embodiments, a gene-editing Cas effector can be a Cas12a nuclease that also makes a double-stranded break in a target DNA sequence. In some embodiments, a gene-editing Cas effector can be a Cas13 nuclease which targets RNA. In some embodiments, a gene-editing Cas effector comprises a Cas9 protein, a Cas13b protein, or a Cas13d protein. In some embodiments, the gene-editing Cas effector comprises a nuclease dead Cas9 (dCas9) protein. In some embodiments, the gene-editing Cas effector comprises a Cas13b protein. In some embodiments, the gene-editing Cas effector comprises a Cas13d protein.

In some embodiments, the gene-editing agent further comprises a guide RNA (gRNA), wherein the gRNA is targeted to an individual gene of a cell. The term “guide RNA” or “gRNA” is a specific type of gRNA that combines tracrRNA (transactivating RNA), which binds to Cas9 to activate the complex to create the necessary strand breaks. and crRNA (CRISPR RNA). comprising complimentary nucleotides to the tracrRNA, into a single RNA construct.

Exemplary methods of employing the CRISPR technique are described in WO 2017/091630, which is incorporated by reference in its entirety.

In some embodiments, the guide RNA can recognize a target RNA. for example. by hybridizing to the target RNA. In some embodiments, the guide RNA comprises a sequence that is complementary to the target RNA. In some embodiments, the gRNA can include one or more modified nucleotides. In some embodiments, the gRNA has a length that is about 10 nt (e.g., about 20 nt, about 30 nt. about 40 nt, about 50 nt. about 60 nt, about 70 nt, about 80 nt, about 90 nt, about 100 nt, about 120 nt, about 140 nt, about 160 nt, about 180 nt. about 200 nt. about 300 nt, about 400 nt, about 500 nt, about 600 nt, about 700 nt, about 800 nt, about 900 nt, about 1000 nt, or about 2000 nt).

In some embodiments, the gene-editing agent comprises a guide RNA (gRNA), wherein the gRNA is targeted to the target gene of the iPSC. In some embodiments, the target gene comprises a COL8A2 gene. In some embodiments, the target gene comprises a L450W mutation in the COL8A2 gene. In some embodiments, the gene-editing agent alters the L450W mutation in the COL8A2 gene. In some embodiments, the gene-editing agent alters the L450W mutation in the COL8A2 gene to a wild-type version of the COL8A2 gene.

In some embodiments, the target gene comprises a LOXHD1 gene. In some embodiments, the target gene comprises a c.1639C>T (p.Arg547Cys) mutation in the LOXHD1 gene. In some embodiments, the gene-editing agent alters the c.1639C>T (p.Arg547Cys) mutation in the LOXHD1 gene. In some embodiments, the gene-editing agent alters the c.1639C>T (p.Arg547Cys) mutation in the LOXHD1gene to a wild-type version of the LOXHD1 gene.

In some embodiments, the target gene comprises a miR-184 gene. In some embodiments, the target gene comprises a +57C>T mutation in the MiR-184 gene. In some embodiments, the gene-editing agent alters the +57C>T mutation in the miR-184 gene. In some embodiments, the gene-editing agent alters the +57C>T mutation in the miR-184 gene to a wild-type version of the miR-184 gene. In some embodiments, a gRNA is targeted to an miR-184 gene. In some embodiments, a gRNA comprises or consists of the sequence shown in SEQ ID NO: 1.

SEQ ID NO: 1-miR-184 gRNA
UUGUGACUGUAAGUGUUGGA

In some embodiments, the gene-editing agent further comprises a PAM sequence. As used herein, the term “photospacer adjacent motif” or “PAM” refers to a sequence that activates the nuclease domain of Cas9. In some embodiments, a PAM-presenting oligonucleotide can include an antisense synthetic oligonucleotide composed of alternating 2′OMe RNA and DNA bases and/or other variations of a PAM presenting oligonucleotide that can optimize the CRISPR/Cas9 system and generate specific cleavage of RNA targets without cross reactivity between non-target RNA or against genomic DNA. In some embodiments, the PAM sequence comprises a TGG sequence.

Specific details regarding the use of CRISPR-Cas systems for genome engineering are described, for example, in Jinek et al., Science 337:816-821 (2012); Cho et al., Nature Biotechnology 31:230-232 (2013): Cong et al., Science 339:819-823 (2013); Jinek et al., eLife 2:e00471 (2013); Mali et al., Science 339:823-826 (2013): Qi et al., Cell 152:1173-1183 (2013): Fu et al., Nature Biotechnology 31:822-826 (2013); Fu et al., Nature Biotechnology 31:822-826 (2013): Hsu et al., Nature Biotechnology 31:827-832 (2013); Mali et al., Nature Biotechnology 31:833-838 (2013): Pattanayak et al., Nature Biotechnology 31:839-843 (2013) and WO/2013/142578. Each of these references in incorporated herein by reference in its entirety.

### Method of Generating an Engineered Corneal Endothelial Cell

Provided herein are methods of generating an engineered corneal endothelial cell, the method including (a) obtaining an induced pluripotent stem cell (iPSC) from a subject; (b) delivering a gene-editing agent into the iPSC, wherein the gene-editing agent alters a target gene of the iPSC; and (c) differentiating the altered iPSC into an engineered corneal endothelial cell.

As used herein, the “comea” refers to the outermost, transparent layer of the eye, and composed of five layers that include the epithelium, Bowman's membrane, stroma, Descemets membrane, and endothelium. The corneal endothelium (CE), is a monolayer of hexagonal cells which is critical in maintaining corneal clarity by mediating hydration through barrier and pump functions. In some embodiments, corneal endothelial dystrophies and surgical trauma can be major factors that contribute to loss of corneal endothelial cells (CECs) and a decrease in CE cell density. Furthermore, Fuchs endothelial corneal dystrophy (FECD) is the leading cause of corneal transplantation.

In some embodiments. iPSCs can offer autologous cell sources for replacement cell therapy, to replace or regenerate tissues by autologous transplantation. In some embodiments, an iPSC can be obtained from a biological sample from a subject. In some embodiments, the iPSC is obtained from blood of the subject. In some embodiments, the iPSC is a peripheral blood mononuclear cell (PBMC) originated iPSC.

Methods of generating engineered corneal endothelial cells are known in the art. Exemplary methods of generating engineered corneal endothelial cells are described in US20220025325, which is incorporated by reference in its entirety For example, United States patent application publication number US20220025325 provides methods of producing corneal endothelial cells (CECs) comprises isolating peripheral blood mononuclear cells (PBMCs) from a biological sample, reprograming the PBMCs to induce pluripotency expressing the pluripotent markers NANOG, OCT4, SOX2, SSEA4, TRA-1-60 and differentiation of the pluripotent stem cells including induced pluripotent stem cells (iPSCs) and human embryonic stem cells (hESCs) to produce corneal endothelial cells (CECs).

Also provided herein, in some embodiments, are uses of the engineered corneal endothelial cell in the manufacture of a medicament for treating an ocular disease in a subject in need thereof. In some embodiments, the subject has been identified as having Fuchs dystrophy.

### EXAMPLES

The disclosure is further described in the following examples, which do not limit the scope of the disclosure described in the claims.

### Example 1—Generation of iPSCs

Cryopreserved PBMCs were reprogramed using a Sendai virus delivery system kit. according to the manufacturer's instructions (Cytotune 2.0; Life Technologies, Carlsbad, CA, USA). The PBMC vial was removed from liquid nitrogen and thawed at 37° C. in a water bath. Subsequently, the PBMCs were washed with medium (StemSpan; STEMCELL Technologies, Inc.) and cultured in StemSpan medium supplemented with 100 ng/mL Feline McDonough Sarcoma-like tyrosine kinase 3 ligand (FLT-3L), 100 ng/mL stem cell factor, 20 ng/mL interleukin-3 (IL-3), and 20 ng/mL interleukin-6 (IL-6), termed complete medium hereafter, in a humidified incubator at 37° C. supplemented with 5% CO2 for 4 days.

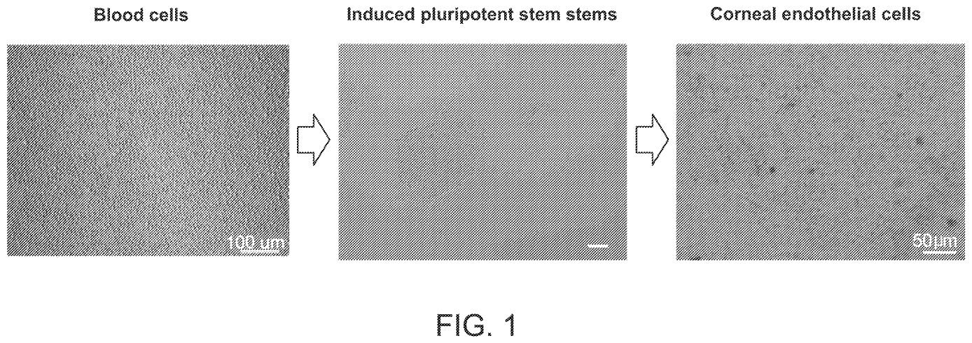

Approximately 5×10⁵ cells/mL PBMCs were collected in a round-bottom tube in 1 mL complete medium, followed by infection by reprogramming viral particles at a multiplicity of infection (MOI) of 5, 5, and 3 (KOS MOI=5: hc-MYC MOI=5; hKLF4 MOI=3). The infected cells were centrifuged at 1000 g for 30 minutes at room temperature. The cells were resuspended in 1 mL complete medium, transferred to a single well of a 12-well plate, and cultured for 3 days in complete medium. Subsequently, the cells were transferred to mouse embryonic fibroblast-coated plates for 3 days with complete medium and finally transferred to Dulbecco's modified Eagle's medium-F12 (DMEM-F12) supplemented with 20% knockout serum replacement (KSR; Life Technologies) and 20 ng/mL basic fibroblast growth factor (bFGF: Life Technologies) until an embryonic stem cell-like colony was formed. Embryonic stem cell-like putative iPSC colonies were selected and cultured on a Matrigel-coated (Corning) plate in mTeSR1 medium (FIG. 1).

### Example 2—CRISPR/Cas9 Gene Editing

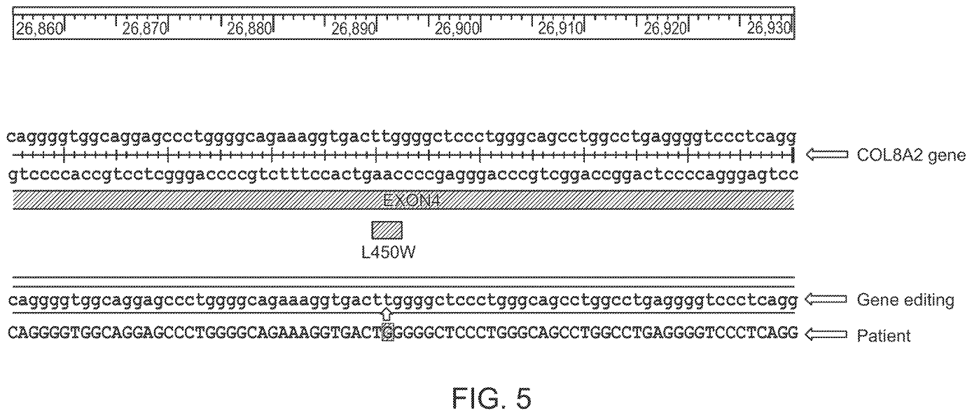

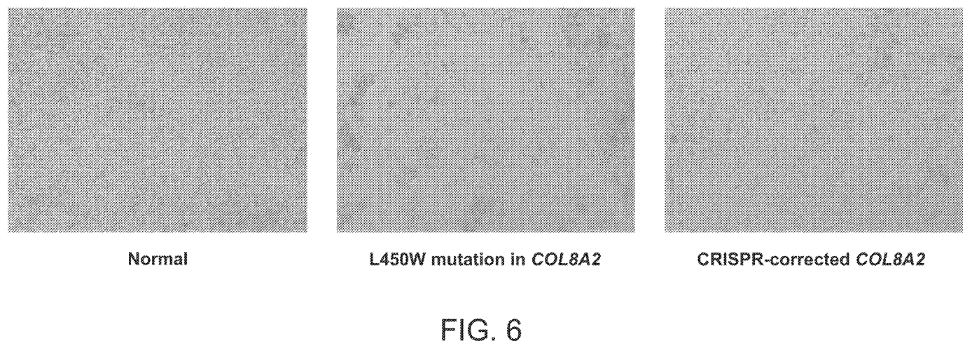

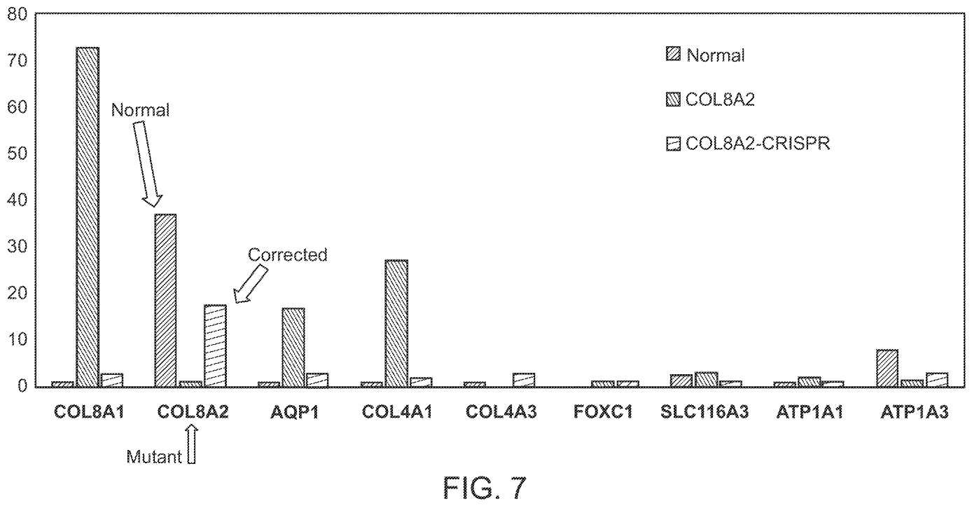

A specific L450W mutation was identified in COL8A2 (NP 001281276.1). The CRISPR/Cas9 gene editing is used to make gene correction in human L450W-COL8A2 iPS cells (FIGS. 4-6). Briefly, the gene sequence is retrieved through HomoloGene (NCBI) and UCSC. For this specific L450W mutation in COL8A2, an evidence-based gRNA designer tool and database DeeoHF (http://www.deephf.com/index/) is used to design 1 to 5 pairs optimized CRISPR guide RNA of gRNAs for proper gene targeting. The gRNA is then ligated into px-gRNA-mCherry vector. Followed by the px-gRNA-mCherry vector construction, the gRNA cutting efficiency is evaluated by co-transfection of pCas9-GFP and px-gRNA-mCherry in 293T cells. A T7 Endonuclease I digestion method is applied to screen the highest indel % efficiency following the standard protocol of NEB T7 Endonuclease I kit (NEB #E3321). After the screen of the gRNA(s) with highest indel efficiency, a 120 bp of single-stranded oligo donor (ssODN) is designed for single-base substitutions by homologous recombination. The ssODN is the reverse sequence of 50 bp of Left arm plus 20 bp of gRNA and 50 bp of Right arm. In order for a quick and cost-effective screen of iPS colonies containing right targeting site, a new restriction enzyme is introduced in the ssODN with no change of the original protein sequence. Human L450W-COL8A2 iPS cells (2×10⁶ cells/line) are then dissociated and transfected with pCas9-GFP, px-mCherry-gRNA and matched ssODN (targeting L450W mutation) using P3 Primary Cell buffer and CB150 program on a 4D-Nucleofector (Lonza, Walkersville). The transfected cells are then plated onto vitronectin-coated plate with mTeSRI medium in the presence of 10 M ROCK inhibitor Y27632. Twenty-four hours after nucleofection, single GFP and mCherrv double-positive cells are sorted into 96-well followed by picking up individual clones and performing PCR and restrictive enzyme screens 7 days after plating. The correct sequence-verified homozygous iPS clone (L450W corrected to right sequence) is saved for the evaluation of final mutation landscapes of base-edited by whole-genome sequencing (WGS).

### Example 3—Differentiation of Corrected iPSCs into Corneal Endothelial Cells

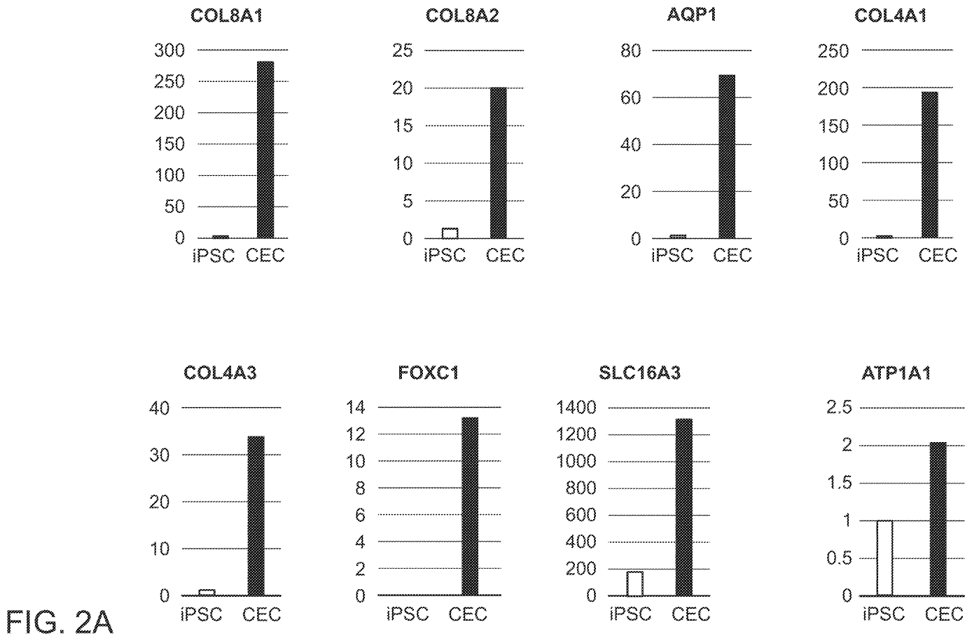

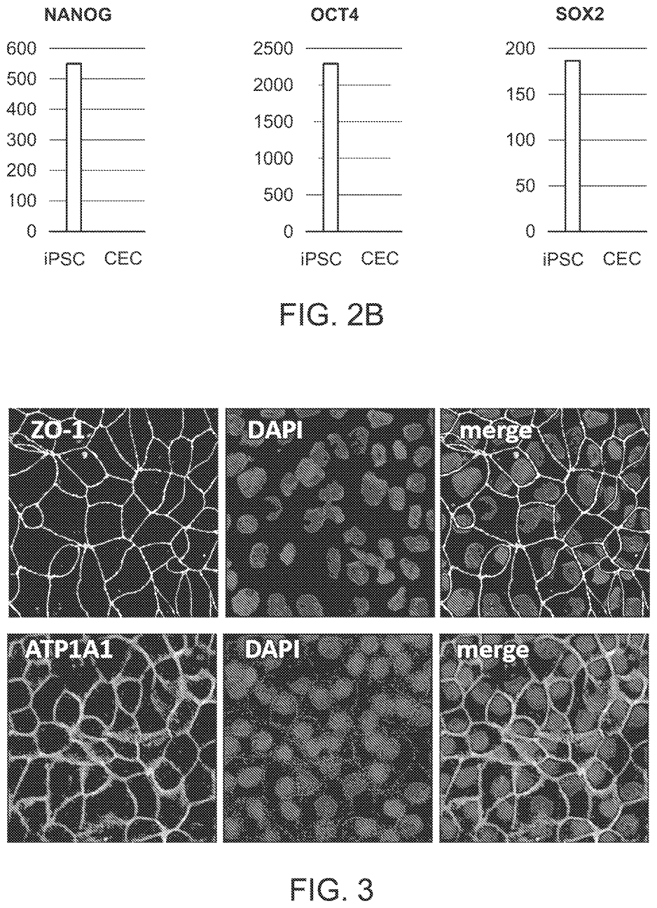

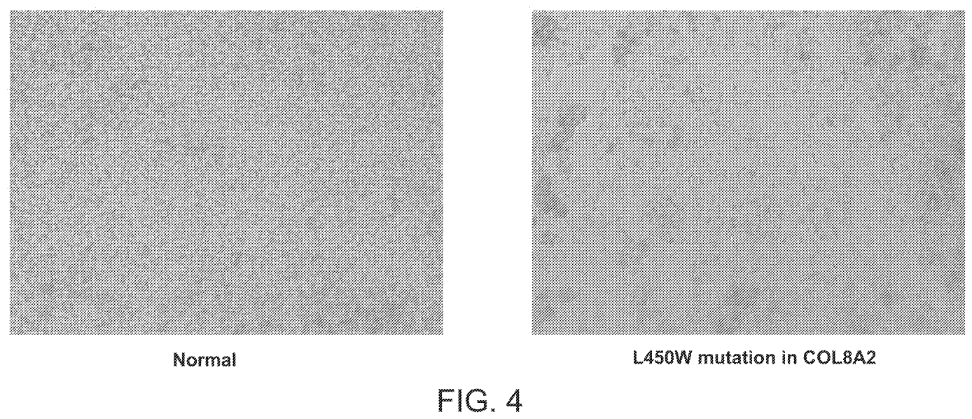

To generate CECs, iPSCs were seeded on 35-mm Matrigel-coated plates (Corning) in 1:12 dilution (80% confluent plate is split into 12 plates) on day 0 using cell dissociation buffer (Life Technologies). The iPSCs were grown for 4 days in medium (mTeSR1; STEMCELL Technologies, Inc.) (FIGS. 2A-2B and 3). On day 4. mTeSRI media was replaced with dual Smad inhibitors media containing 500 ng/mL human recombinant Noggin (R&D Systems, Minneapolis, MN, USA) and 10 M SB431542 (MilliporeSigma) in a basal media of 80% DMEM-F12 (Life Technologies), 20% KSR (Life Technologies), 1% nonessential amino acids (Life Technologies), 1 mM 1-glutamine (STEMCELL Technologies, Inc.), 0.1 mM β-mercaptoethanol (MilliporeSigma), and 8 ng/mL bFGF (MilliporeSigma).

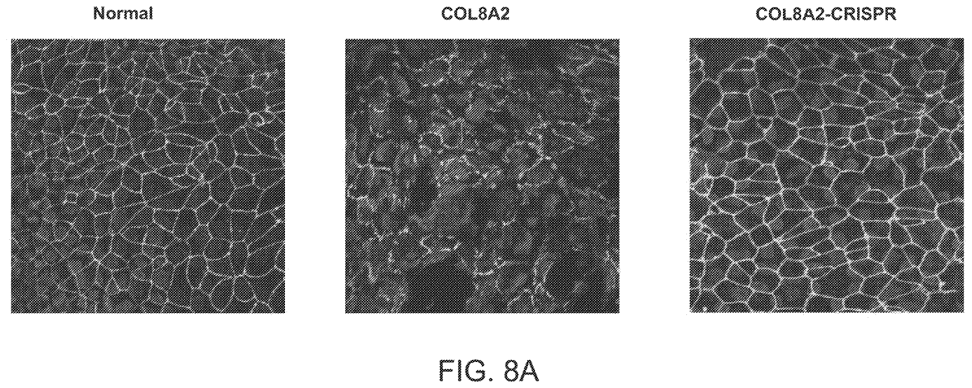

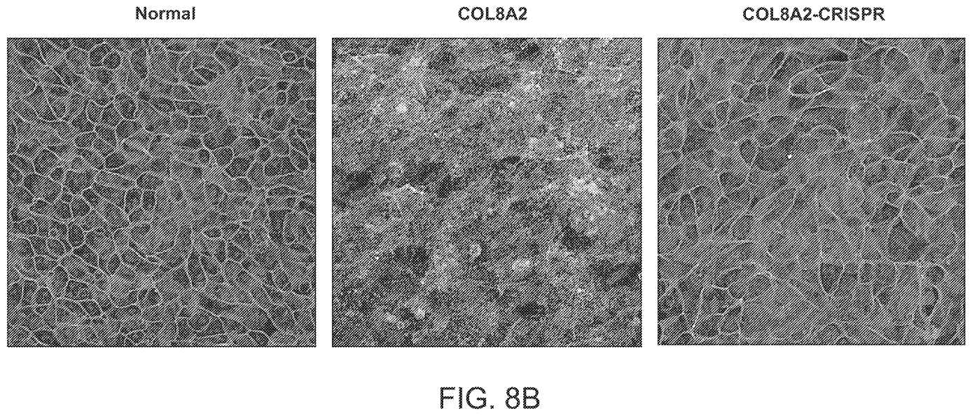

On day 6, dual Smad inhibitors media was replaced by comea medium containing 0.1×B27 supplement (Life Technologies), 10 ng/mL recombinant human platelet derived growth factor-BB (PDGF-BB: PeproTech, Rocky Hill, NJ, USA), and 10 ng/mL recombinant human Dickkopf related protein-2 (DKK-2; R&D Systems) in a basal media of 80% DMEM-F12 (Life Technologies), 20% KSR (Life Technologies), 1% nonessential amino acids (Life Technologies), 1 mM 1-glutamine (STEMCELL Technologies, Inc.), 0.1 mM 0-mercaptoethanol (MilliporeSigma), and 8 ng/mL bFGF (MilliporeSigma). On day 7, the differentiating CECs were transferred to new Matrigel-coated plates (35 mm) and were grown in cornea medium for 13 additional days (FIGS. 7 and 8A-8B).

What is claimed is:

1. A method of treating an ocular disease in a subject in need thereof, the method comprising:
(a) obtaining an induced pluripotent stem cell (iPSC) from the subject;
(b) delivering a gene-editing agent into the iPSC, wherein the gene-editing agent alters a target gene of the iPSC;
(c) differentiating the altered iPSC into a corneal endothelial cell; and
(d) administering the corneal endothelial cell to the subject, thereby treating the ocular disease in the subject.

2. The method of claim 1, wherein the iPSC is obtained from blood of the subject.

3. The method of claim 1 or 2, wherein the iPSC is a peripheral blood mononuclear cell (PBMC) originated iPSC.

4. The method of any one of claims 1-3, wherein the gene-editing agent comprises CRISPR/Cas9 components.

5. The method of claim 4, wherein the gene-editing agent comprises a guide RNA (gRNA), wherein the gRNA is targeted to the target gene of the iPSC.

6. The method of any one of claims 1-5, wherein the target gene comprises a COL8A2 gene.

7. The method of claim 6, wherein the target gene comprises a L450W mutation in the COL8A2 gene.

8. The method of claim 7, wherein the gene-editing agent alters the L450W mutation in the COL8A2 gene.

9. The method of any one of claims 1-8, wherein the gene-editing agent further comprises a PAM sequence.

10. The method of claim 9, wherein the PAM sequence comprises a TGG sequence.

11. The method of any one of claims 1-10, wherein the ocular disease comprises Fuchs endothelial corneal dystrophy (FECD), keratoconus, keratoconjunctivitis sicca (KCS), hepes virus infections, varicella-zoster virus infections, irido-corneal endothelial syndrome (ICE), pterygium, Stevens Johnson Syndrome (SJS), corneal ulcers, or bullous keratopathy.

12. The method of claim 11, wherein the ocular disease is Fuchs dystrophy.

13. A method of generating an engineered corneal endothelial cell, the method comprising:
(a) obtaining an induced pluripotent stem cell (iPSC) from a subject;
(b) delivering a gene-editing agent into the iPSC, wherein the gene-editing agent alters a target gene of the iPSC; and
(c) differentiating the altered iPSC into an engineered corneal endothelial cell.

14. The method of claim 13, wherein the iPSC is obtained from blood of the subject.

15. The method of claim 13 or 14, wherein the iPSC is a peripheral blood mononuclear cell (PBMC) originated iPSC.

16. The method of any one of claims 13-15, wherein the gene-editing agent comprises CRISPR/Cas9 components.

17. The method of claim 16, wherein the gene-editing agent comprises a guide RNA (gRNA), wherein the gRNA is targeted to the target gene of the iPSC.

18. The method of any one of claims 13-17, wherein the target gene comprises a COL8A2 gene.

19. The method of claim 18, wherein the target gene comprises a L450W mutation in the COL8A2 gene.

20. The method of claim 19, wherein the gene-editing agent alters the L450W mutation in the COL8A2 gene.

21. The method of any one of claims 13-20, wherein the gene-editing agent further comprises a PAM sequence.

22. The method of claim 21, wherein the PAM sequence comprises a TGG sequence.
